(19) United States
(12) Patent Application Publication
(10) Pub. No.: US 2023/0290447 A1
(43) Pub. Date: Sep. 14, 2023

(54) SYSTEMS AND METHODS FOR CONDUCTING EYE MOVEMENT DESENSITIZATION AND REPROCESSING

(71) Applicant: Innovative Bilateral Designs LLC, White Plains, NY (US)

(72) Inventor: Lisa LaRosa, Litchfield, CT (US)

(73) Assignee: Innovative Bilateral Designs LLC, White Plains, NY (US)

(21) Appl. No.: 17/654,267

(22) Filed: Mar. 10, 2022

(51) Int. Cl. G16H 10/20; G16H 20/70

(52) U.S. Cl. CPC G16H 10/20; G16H 20/70

(57) ABSTRACT

Disclosed herein are systems and methods for conducting eye movement desensitization and reprocessing. Systems and methods may include connecting a session master to a session, creating a room, connecting a session guest to the session, configuring the room, conducting a baseline screening, conducting a session set, and conducting a post-session screening.

BACKGROUND

A form of psychotherapy, eye movement desensitization and reprocessing (EMDR) may be a form of treatment for clients who may exhibit negative thoughts, feelings, and behaviors, which may result from unprocessed memories (e.g., clients who suffer from post-traumatic stress disorder (PTSD)). The basic function of EMDR is for a client to be directed through a bilateral simulation (e.g., using side-to-side rapid eye movement or hand tapping) while recalling distressing images. Standard EMDR may involve developing a client's dual (e.g., simultaneous) attention to traumatic thoughts, images, or sensations and bilateral stimulation as described above. In EMDR, such dual attention may provide for a mental state suitable for mental reprocessing, which may help in overcoming the negative thoughts, feelings, or behaviors resulting from unprocessed memories.

Treating children or adolescent using EMDR presents specific challenges. It has been found that children and adolescents are less susceptible to treatment using virtual EMDR because they are historically have been unable to visually track in a way that facilitates treatment. A particular need exists thus exists for EMDR treatment options for with children and adolescents.

### SUMMARY

In embodiments of the invention, a method comprises connecting a session master to a session; creating a room; connecting a session guest to the session; configuring the room; conducting a baseline screening; conducting a session set; and conducting a post-session screening.

In further embodiments, the step of configuring the room is performed with both the session master and the session guest. In still further embodiments, the step of conducting the baseline screening includes determining an emotional level for a client. In still further embodiments, the room comprises a foreground object and a background, and wherein configuring the room includes configuring a motion path of the foreground object relative to the background. In still further embodiments, the motion path of the foreground object relative to the background includes a horizontal motion path, a corner path motion path, a figure-eight horizontal motion path, a figure-eight vertical motion path, a diagonal motion path, or a vertical motion path.

### BRIEF DESCRIPTION OF THE DRAWINGS

For a fuller understanding of the nature and objects of the disclosure, reference should be made to the following detailed description taken in conjunction with the accompanying drawings, in which:

FIG. 1 illustrates a system for conducting EMDR, according to one or more embodiments;

FIG. 2 illustrates a method for conducting EMDR, according to one or more embodiments;

FIG. 3 illustrates a room creation screen, according to one or more embodiments;

FIG. 4 illustrates a room name input screen, according to one or more embodiments;

FIG. 5 illustrates an introductory screen, according to one or more embodiments;

FIG. 6 illustrates a room selection screen, according to one or more embodiments;

FIG. 7 illustrates a selection screen, according to one or more embodiments;

FIG. 8 illustrates a background selection screen, according to one or more embodiments;

FIG. 9 illustrates a foreground object selection screen, according to one or more embodiments;

FIG. 10 illustrates a session configuration screen, according to one or more embodiments;

FIG. 11 illustrates an emotion input screen, according to one or more embodiments;

FIG. 12 illustrates an emotion input screen, according to one or more embodiments;

FIG. 13 illustrates an emotion location screen, according to one or more embodiments;

FIG. 14 illustrates a configuration screen, according to one or more embodiments;

FIG. 15 illustrates an in-session screen, according to one or more embodiments;

FIG. 16 illustrates an in-session screen, according to one or more embodiments;

and

FIG. 17 illustrates a lobby screen, according to one or more embodiments.

### DETAILED DESCRIPTION

It is to be understood that the invention is not limited in its application to the details of construction and the arrangements of components and/or method steps set forth in the following description or illustrated in the drawings, and phraseology and terminology used herein is for the purpose of description and should not be regarded as limiting. The invention is capable of other embodiments and of being practiced or being carried out in various ways. Accordingly, other aspects, advantages, and modifications will be apparent to those skilled in the art to which the invention pertains, and these aspects and modifications are within the scope of the invention.

Embodiments disclosed herein may include systems and methods for conducting EMDR. An application server may be used to provide for conducting an EMDR therapy session by a therapist as a session master and a client as a session guest, where the therapist may control various aspects of the EMDR session and visualize the client in real-time.

Embodiments herein may provide systems and methods for use by EMDR therapists in performing or training for performing EMDR. Such embodiments may provide multiple creative forms of bilateral stimulation to achieve a state of dual attention. Visually appealing designs may help children, adolescents and adults maintain focus and attention on present awareness, while their EMDR therapist facilitates the processing of past memories.

Embodiments may provide a split-screen telehealth platform for clients to be seen and guided by an EMDR therapist while engaging with the application. Such embodiments may enable EMDR clinicians to conduct EMDR remotely by providing a visually appealing game-like design to help clients maintain a state of dual-awareness by visually tracking objects moving bilaterally across the screen. Embodiments disclosed herein may be of particular use to clients, especially children and teenagers who are unable to attain a state of dual awareness by visually tracking other less creative or visually engaging designs that are not interactive in nature.

Interactive components may be integrated to offer an additional means of achieving dual attention through the use of tactile simulation. Further simulations may include storybooks, which may help clients engage in eye movements and bilateral movements while reading therapeutic stories.

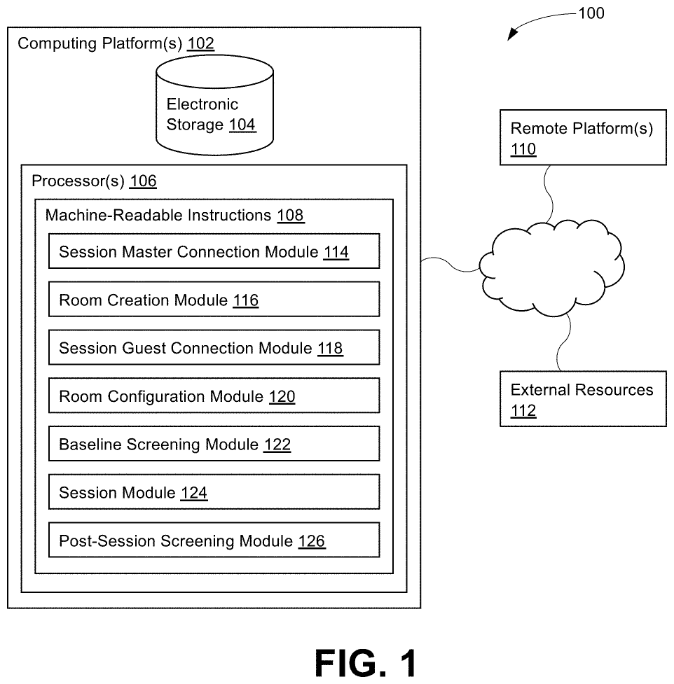

FIG. 1 illustrates a system 100 for conducting EMDR according to one or more embodiments. In some embodiments, system 100 may include one or more computing platforms 102. Computing platform(s) 102 may be configured to communicate with one or more remote platforms 110 according to a client/server architecture, a peer-to-peer architecture, and/or other architectures. Remote platform(s) 110 may be configured to communicate with other remote platforms via computing platform(s) 102 and/or according to a client/server architecture, a peer-to-peer architecture, and/or other architectures. Users may access system 100 via remote platform(s) 110.

Computing platform(s) 102 may be configured by machine-readable instructions 108. Machine-readable instructions 108 may include one or more instruction modules. The instruction modules may include computer program modules. The instruction modules may include one or more of a session master connection module 114, a room creation module 116, a session guest connection module 118, a room configuration module 120, a baseline screening module 122, a session module 124, and a post-session screening module 126, and/or other instruction modules.

Session master connection module 114 may include an application interface configured to enable a session master (e.g., a therapist) to a session on an application server.

Room creation module 116 may include an application interface configured to enable a session master to create a room. Using room creation module 116 may enable the therapist to set a room name and a maximum number of players. In some embodiments, there may be one client, and in others there may be more than one client (e.g., up to eight (or more) clients).

Session guest connection module 118 may include an application interface, which may enable a session guest (e.g., a client) to connect to the application server. A client may be prohibited from creating a session (e.g., a client may only join a session).

Room configuration module 120 may include an application interface, which may enable a session master to configure the session. The session master may, with or without the session guest, select a background (e.g., a black screen, a starry night sky, a starry night mountain, an ocean, a flower meadow, etc.) and a foreground object (e.g., a UFO, a beach ball, a firefly, etc.). Selection of the foreground objects and background may enable an ability to customize the session to enhance various aspects for the client (e.g., engagement, relaxation, etc.).

Baseline screening module 122 may include confirming, according to EMDR protocol, whether the client is ready, willing, and able to discuss the situation of interest. The therapist may utilize the baseline screening to configure the bilateral motion of the object. Such screening may include determining a client's baseline stress or distress level. Such baseline screening may include a determination of a rating of feelings regarding the situation of interest (e.g., using emojis or a scale (e.g., 1-10) or a rating for each of various feelings), where the client feels a particular feeling in their body (e.g., via an on-screen selection). Additional procedural EMDR questions may be asked by the therapist. Parameters of bilateral motion of the object may include determine a session time, a foreground object speed, a foreground object size, a foreground object rotation speed (e.g., the rate at which the foreground object turns into and/or away from the main plane of the screen), a foreground object spawn position (e.g., a size of the window the foreground object appears within and whether the foreground object spawns on the screen (e.g., top, middle, or bottom)), a foreground object variance, and a foreground object motion path (e.g., horizontal, corner path (e.g., hourglass), figure-eight horizontal, figure-eight vertical, diagonal, or vertical). After selection of the background and the foreground object, and configuration of the bilateral motion of the foreground object, the therapist and client may begin the session.

Session set conducting module 124 may include an application interface to conduct EMDR sessions between a therapist as the session master and a client as the session guest. One or more sets may be conducted in the session. The therapist may be able to observe the client during each set and adjust in real-time as necessary. For example, between sets, the therapist may determine with the client what aspect of the situation of interest the client is noticing, and then may tailor the parameters of the next set to focus on that aspect of the situation of interest.

The motion of the foreground object may be three-dimensional (e.g., the foreground object, in addition to moving in the plane of the screen may appear to move “into” and “out from” the screen). In-session, the therapist presentation (e.g., the session master) may include the background, the foreground object, in-session parameter controls (e.g., such that the therapist may make changes to the parameters of the session in real-time), and a video feed of the client (e.g., through the client's webcam). By having the ability to see the client, the therapist may be able to determine the client's reactions or other visual responses, as well as track the client's eye movement. Such real-time observation may be used by a therapist to adjust the session and the parameters of the session in real-time. Various settings may be configured along scales, for example, 1-10 or 1-100. In-session, the client presentation (e.g., the session guest) may include the background and the foreground object. A client may be challenged to track movement of the foreground object from one side of the screen to the other. Sound may accompany the movement (e.g., a buzzing sound may accompany movement of a firefly). The session may be run from an application server, which may perform in a platform-as-a-service delivery environment (e.g., Photon Cloud, GameSparks, Unity, or PlayFab). Tactile feedback may be incorporated into the session. The visual elements presented to the client may be interactive and responsive to input from the client or clinician. The visual elements may be presented to appear three-dimensional or be presented in a virtual reality environment.

In embodiments of the invention, the clinician may have complete maneuverability of the foreground object to enable movement in a novel directional path of the clinician's choosing should the need arise (e.g., if the processing becomes stuck or stalled).

Post-session screening module 126 may include an application interface for the session master to input post-session data regarding the client. A final check-in, which may include determining a Subjective Units of Distress scale (SUDs) rating, may be performed to determine the results of the session.

In some embodiments, computing platform(s) 102, remote platform(s) 110, and/or external resources 112 may be operatively linked via one or more electronic communication links. For example, such electronic communication links may be established, at least in part, via a network such as the Internet and/or other networks using, for example, TCP/IP or cellular hardware enabling wired or wireless communication. It will be appreciated that this is not intended to be limiting, and that the scope of this disclosure includes embodiments in which computing platform(s) 102, remote platform(s) 110, and/or external resources 112 may be operatively linked via some other communication media.

A given remote platform 110 may include one or more processors configured to execute computer program modules. The computer program modules may be configured to enable an expert or user associated with the given remote platform 110 to interface with system 100 and/or external resources 112, and/or provide other functionality attributed herein to remote platform(s) 110. By way of non-limiting example, a given remote platform 110 and/or a given computing platform 102 may include one or more of a server, a desktop computer, a laptop computer, a handheld computer, a tablet computing platform, a NetBook, a Smartphone, a gaming console, and/or other computing platforms.

External resources 112 may include sources of information outside of system 100, external entities participating with system 100, and/or other resources. In some embodiments, some or all of the functionality attributed herein to external resources 112 may be provided by resources included in system 100.

Computing platform(s) 102 may include electronic storage 104, one or more processors 106, and/or other components. Computing platform(s) 102 may include communication lines, or ports to enable the exchange of information with a network and/or other computing platforms. Illustration of computing platform(s) 102 in FIG. 1 is not intended to be limiting. Computing platform(s) 102 may include a plurality of hardware, software, and/or firmware components operating together to provide the functionality attributed herein to computing platform(s) 102. For example, computing platform(s) 102 may be implemented by a cloud of computing platforms operating together as computing platform(s) 102.

Electronic storage 104 may comprise non-transitory storage media that electronically stores information. The electronic storage media of electronic storage 104 may include one or both of system storage that is provided integrally (i.e., substantially non-removable) with computing platform(s) 102 and/or removable storage that is removably connectable to computing platform(s) 102 via, for example, a port (e.g., a USB port, a firewire port, etc.) or a drive (e.g., a disk drive, etc.). Electronic storage 104 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 104 may include one or more virtual storage resources (e.g., cloud storage, a virtual private network, and/or other virtual storage resources). Electronic storage 104 may store software algorithms, information determined by processor(s) 106, information received from computing platform(s) 102, information received from remote platform(s) 110, and/or other information that enables computing platform(s) 102 to function as described herein.

Processor(s) 106 may be configured to provide information processing capabilities in computing platform(s) 102. As such, processor(s) 106 may include one or more of a digital processor, an analog processor, a digital circuit designed to process information, an analog circuit designed to process information, a state machine, and/or other mechanisms for electronically processing information. Although processor(s) 106 is shown in FIG. 1 as a single entity, this is for illustrative purposes only. In some embodiments, processor(s) 106 may include a plurality of processing units. These processing units may be physically located within the same device, or processor(s) 106 may represent processing functionality of a plurality of devices operating in coordination. Processor(s) 106 may be configured to execute modules 114, 116, 118, 120, 122, 124, 126, and/or other modules. Processor(s) 106 may be configured to execute modules 114, 116, 118, 120, 122, 124, 126, and/or other modules by software; hardware; firmware; some combination of software, hardware, and/or firmware; and/or other mechanisms for configuring processing capabilities on processor(s) 106. As used herein, the term “module” may refer to any component or set of components that perform the functionality attributed to the module. This may include one or more physical processors during execution of processor readable instructions, the processor readable instructions, circuitry, hardware, storage media, or any other components. Various modules or portions thereof may be implemented in any of various ways, including procedure-based techniques, component-based techniques, and/or object-oriented techniques, among others. For example, the program instructions may be implemented using ActiveX controls, C++ objects, JavaBeans, Microsoft Foundation Classes (MFC), Streaming SIMD Extension (SSE), or other technologies or methodologies, as desired.

It should be appreciated that although modules 114, 116, 118, 120, 122, 124, and 126 are illustrated in FIG. 1 as being implemented within a single processing unit, in embodiments in which processor(s) 106 includes multiple processing units, one or more of modules 114, 116, 118, 120, 122, 124, or 126 may be implemented remotely from the other modules. The description of the functionality provided by the different modules 114, 116, 118, 120, 122, 124, and 126 described below is for illustrative purposes, and is not intended to be limiting, as any of modules 114, 116, 118, 120, 122, 124, or 126 may provide more or less functionality than is described. For example, one or more of modules 114, 116, 118, 120, 122, 124, or 126 may be eliminated, and some or all of its functionality may be provided by other ones of modules 114, 116, 118, 120, 122, 124, or 126. As another example, processor(s) 106 may be configured to execute one or more additional modules that may perform some or all of the functionality attributed below to one of modules 114, 116, 118, 120, 122, 124, or 126.

Communications between various components and/or modules of system 100, including between one or more of computing platform 102 (including the various components and/or modules thereof), remote platform 110, and external resources 112 may include communications and/or transfer of data via the internet.

The internet may include an interconnected network of systems and a suite of protocols for the end-to-end transfer of data therebetween. A model describing may be the Transport Control Protocol and Internet Protocol (TCP/IP), which may also be referred to as the internet protocol suite. TCP/IP provides a model of four layers of abstraction: an application layer, a transport layer, an internet layer, and a link layer. The link layer may include hosts accessible without traversing a router, and thus may be determined by the configuration of the network (e.g., a hardware network implementation, a local area network, a virtual private network, or a networking tunnel). The link layer may be used to move packets of data between the internet layer interfaces of different hosts on the same link. The link layer may interface with hardware for end-to-end transmission of data. The internet layer may include the exchange of datagrams across network boundaries (e.g., from a source network to a destination network), which may be referred to as routing, and is performed using host addressing and identification over an internet protocol (IP) addressing system (e.g., IPv4, IPv6). A datagram may include a self-contained, independent, basic unit of data, including a header (e.g., including a source address, a destination address, and a type) and a payload (e.g., the data to be transported), to be transferred across a packet-switched network. The transport layer may utilize the user datagram protocol (UDP) to provide for basic data channels (e.g., via network ports) usable by applications for data exchange by establishing end-to-end, host-to-host connectivity independent of any underlying network or structure of user data. The application layer may include various user and support protocols used by applications users may use to create and exchange data, utilize services, or provide services over network connections established by the lower layers, including, for example, routing protocols, the hypertext transfer protocol (HTTP), the file transfer protocol (FTP), the simple mail transfer protocol (SMTP), and the dynamic host configuration protocol (DHCP). Such data creation and exchange in the application layer may utilize, for example, a client-server model or a peer-to-peer networking model. Data from the application layer may be encapsulated into UDP datagrams or TCP streams for interfacing with the transport layer, which may then effectuate data transfer via the lower layers.

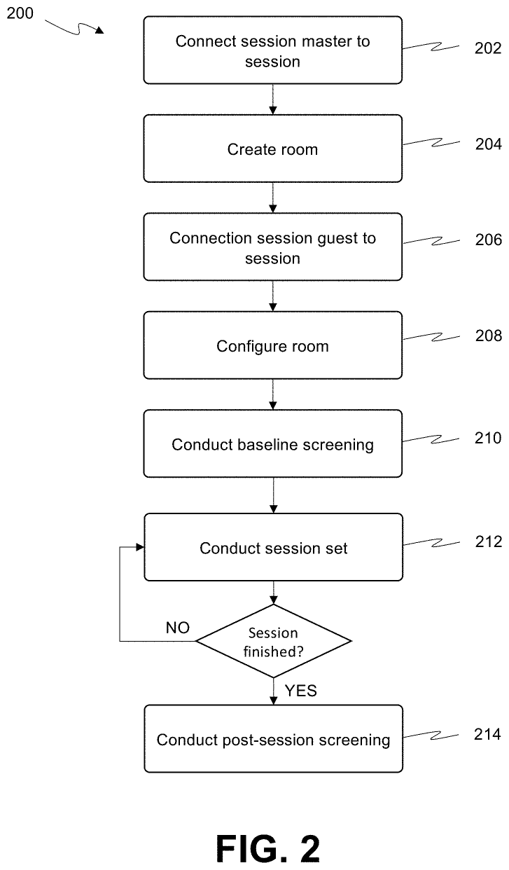

FIG. 2 illustrates a method 200 for conducting EMDR, according to one or more embodiments. The operations of method 200 presented below are intended to be illustrative. In some embodiments, method 200 may be accomplished with one or more additional operations not described, and/or without one or more of the operations discussed. Additionally, the order in which the operations of method 200 are illustrated in FIG. 2 and described below is not intended to be limiting.

In some embodiments, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include connecting a session master to a session. Operation 202 may be performed by one or more hardware processors configured by machine-readable instructions including a module that is the same as or similar to session master connection module 114, in accordance with one or more embodiments.

An operation 204 may include creating a room. Operation 204 may be performed by one or more hardware processors configured by machine-readable instructions including a module that is the same as or similar to room creation module 116, in accordance with one or more embodiments.

An operation 206 may include connecting a session guest to the session. Operation 206 may be performed by one or more hardware processors configured by machine-readable instructions including a module that is the same as or similar to session guest connection module 118, in accordance with one or more embodiments.

An operation 208 may include configuring a room. Operation 208 may be performed by one or more hardware processors configured by machine-readable instructions including a module that is the same as or similar to room configuration module 120, in accordance with one or more embodiments.

An operation 210 may include conducting a baseline screening. Operation 210 may be performed by one or more hardware processors configured by machine-readable instructions including a module that is the same as or similar to baseline screening module 122, in accordance with one or more embodiments.

An operation 212 may include conducting a session set. Operation 212 may be performed one or multiple times during the course of a session. Operation 212 may be performed by one or more hardware processors configured by machine-readable instructions including a module that is the same as or similar to session module 124, in accordance with one or more embodiments.

An operation 214 may include conducting a post-session screening. Operation 214 may be performed by one or more hardware processors configured by machine-readable instructions including a module that is the same as or similar to post-session screening module 126, in accordance with one or more embodiments.

Various steps, functions, and/or operations of the computing platform(s) 102, the remote platform(s) 110, and/or the external resources 112 and the methods disclosed herein may be carried out by one or more of, for example, electronic circuits, logic gates, multiplexers, programmable logic devices, ASICs, analog or digital controls/switches, microcontrollers, or computing systems. Program instructions implementing methods such as those described herein may be transmitted over or stored on carrier medium. The carrier medium may include a storage medium such as a read-only memory, a random access memory, a magnetic or optical disk, a non-volatile memory, a solid state memory, a magnetic tape, and the like. A carrier medium may include a transmission medium such as a wire, cable, or wireless transmission link. For instance, the various steps described throughout the present disclosure may be carried out by a single processor 106 (or computing platform 102) or, alternatively, multiple processors 106 (or multiple computing platforms 102). Moreover, different sub-systems of the system 100 may include one or more computing or logic systems. Therefore, the above description should not be interpreted as a limitation on the present disclosure but merely an illustration.

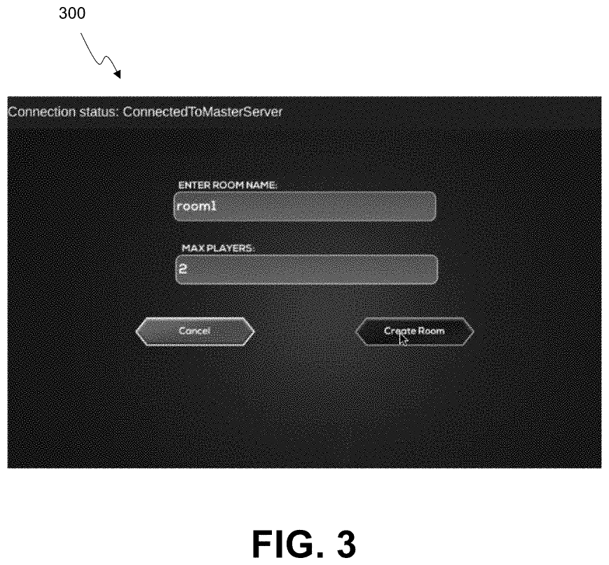

FIG. 3 illustrates a room creation screen 300, according to one or more embodiments. Room creation screen 300 may be used to create a room. Room creation screen 300 may provide controls to a session master, which may be used to define parameters of a room, for example, a room name and a maximum number of players. Room creation screen 300 may further provide an option for the session master to cancel the room creation. Room creation screen 300 may further provide the session master with an option to create the room.

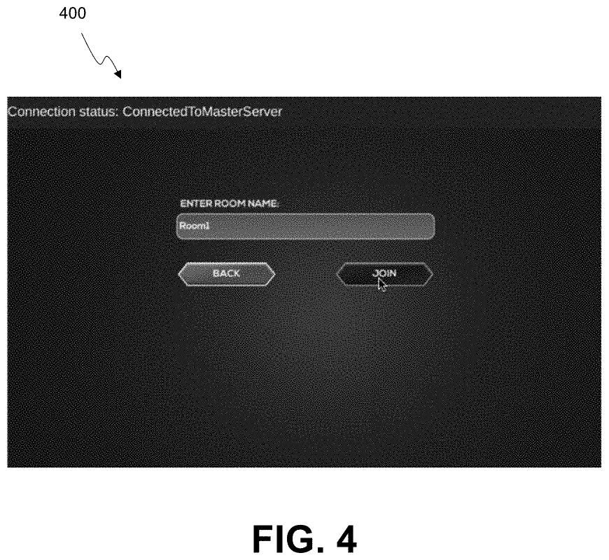

FIG. 4 illustrates a room name input screen 400, according to one or more embodiments. Room name input screen 400 may be used by, for example, a session guest to select the room into which the session guest will enter. Room name input screen 400 may present the session guest with the ability to enter the room name or select a room name. Room name input screen 400 may further provide the session guest with an ability to return to a previous screen, or to join the room.

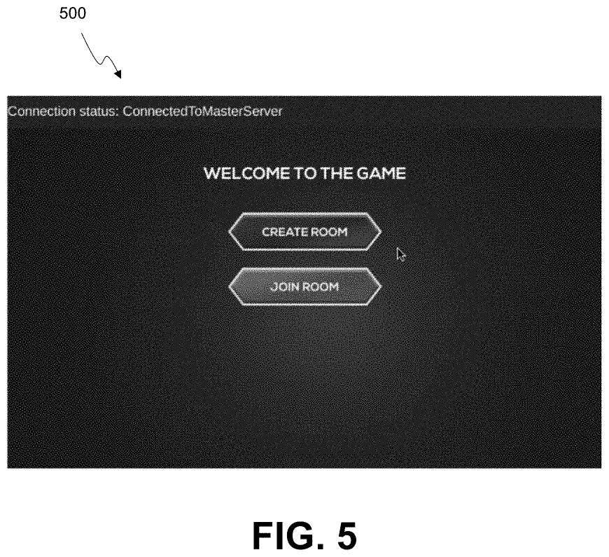

FIG. 5 illustrates an introductory screen 500, according to one or more embodiments. Introductory screen 500 may present to a user options to create a room or to join a room.

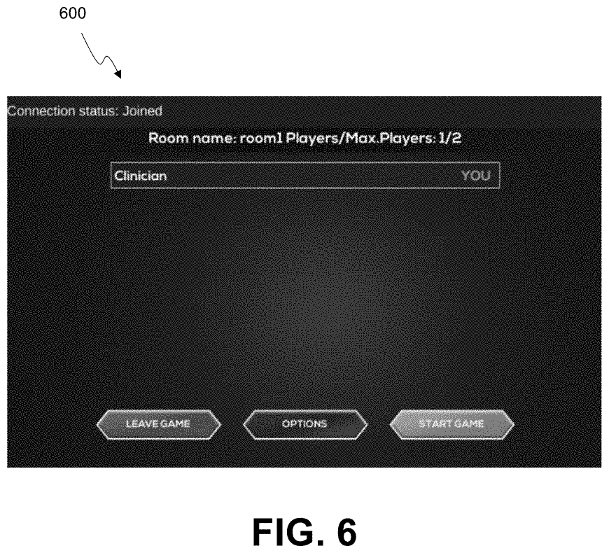

FIG. 6 illustrates a room selection screen 600, according to one or more embodiments. Room selection screen 600 may be used by, for example, a session master or a session guest to select a room into which the session master or the session guest will enter. Room selection screen 600 may present the session guest with the ability to enter the room name or select a room name. Room selection screen 600 may further provide the session guest with an ability to return to a previous screen, or to join the room.

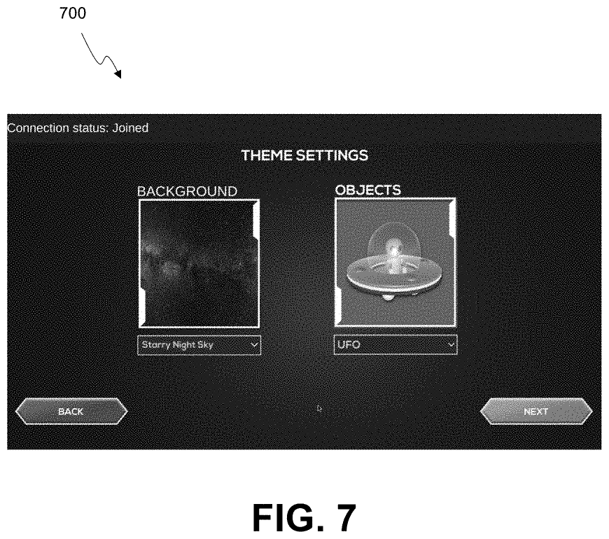

FIG. 7 illustrates a selection screen 700, according to one or more embodiments. Selection screen 700 may present a session master and/or a session guest with an ability to select a background and a foreground object.

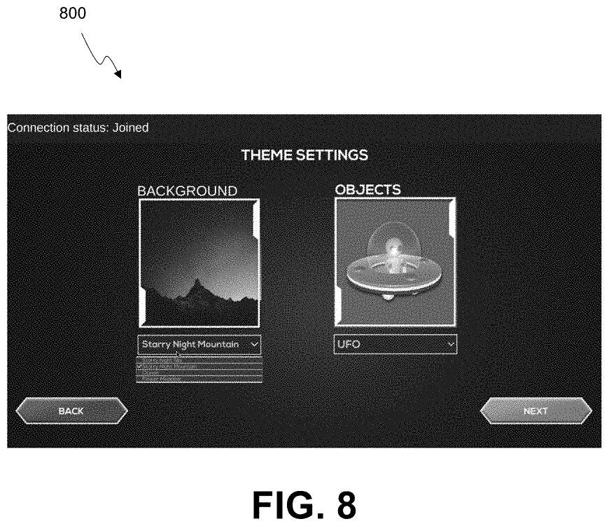

FIG. 8 illustrates a background selection screen 800, according to one or more embodiments. Background selection screen 800 may include options for which the session master, for example, with the help of the session guest, may select a background. For example, a selected background may include an image of, inter alia, a stary night mountain, a stary night sky, an ocean, or a flower meadow.

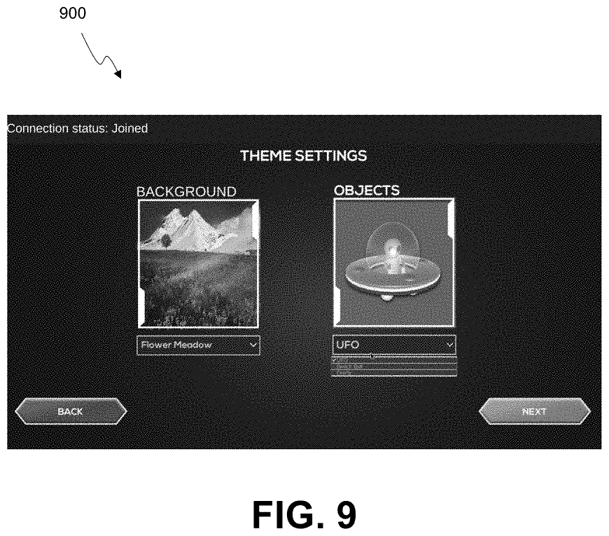

FIG. 9 illustrates a foreground object selection screen 900, according to one or more embodiments. Foreground object selection screen 900 may include options for which the session master, for example, with the help of the session guest, may select a foreground object. For example, a selected foreground object may include an image of, inter alia, a UFO, a beach ball, or a firefly.

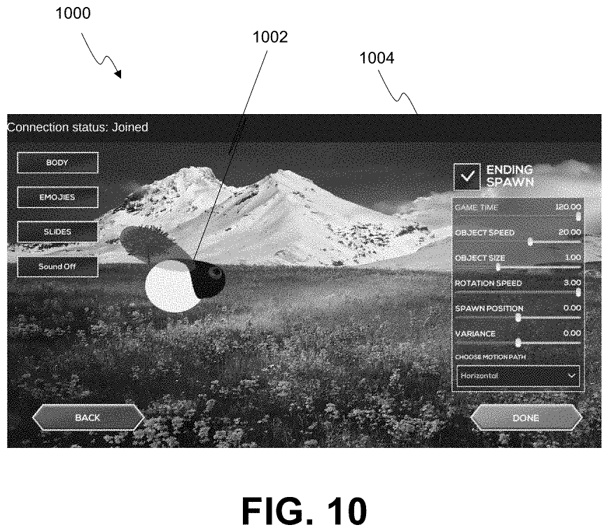

FIG. 10 illustrates a session configuration screen 1000, according to one or more embodiments. Session configuration screen 1000 may present a foreground object 1002 overlayed on a background 1004. Various configuration options of the motion of foreground object 1002 may be presented on session configuration screen 1000 for configuration by the session master. Such options may include, for example, an ending spawn location, a session time, an object speed, an object size, a rotation speed, a spawn position, a variants, and a motion path. Various of the settings may be selected by the EMDR therapist based on predetermined parameters, findings, medical knowledge, technical expertise, in-session observations, baseline screening, or other appropriate configuration drivers. Session configuration screen 1000 may additionally present to the session master options for performing a baseline screening, an in-session screening, or a post-session screening. Such screening options may include screenings related to the clients body, a motion, or other aspects of the client in relation to the EMDR session. Within the session configuration screen 1000, foreground object 1002 may be frozen while configurations or screenings are being performed, or foreground object 1002 may be in motion as configured by the options of session configuration screen 1000.

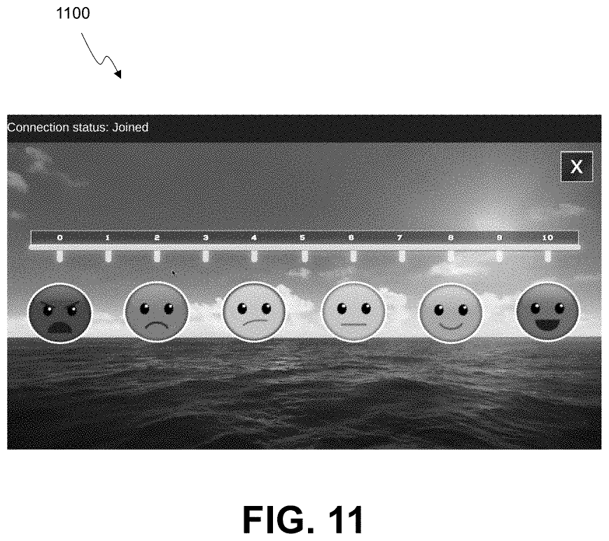

FIG. 11 illustrates an emotion input screen 1100, according to one or more embodiments. Emotion input screen 1100 may present to a session master or a session guest an ability to select regarding the clients emotional state. For example, emotion input screen 1100 may present a scale, for example, from zero to ten, corresponding to potential emotional states of the client. Various of the values of the scale may be associated with a corresponding description of the described emotional state, or, in some embodiments, an emoji.

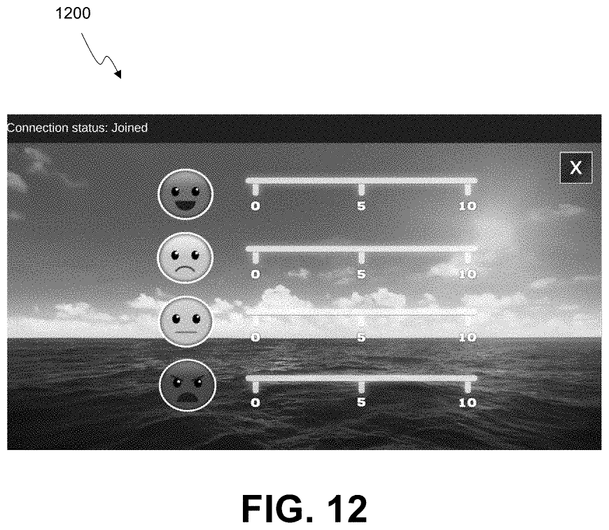

FIG. 12 illustrates an emotion input screen 1200, according to one or more embodiments. Emotion input screen 1200 may present to the session master or session guest an ability to select levels of various emotions the client is experiencing. For example, each of happiness, sadness, numbness, emotionlessness, ambivalence, anger, frustration, or other emotions may be represented on emotion input screen 1200, each with a corresponding selector, using which the session master or the session guest may be able to input levels describing the emotions experienced by the client.

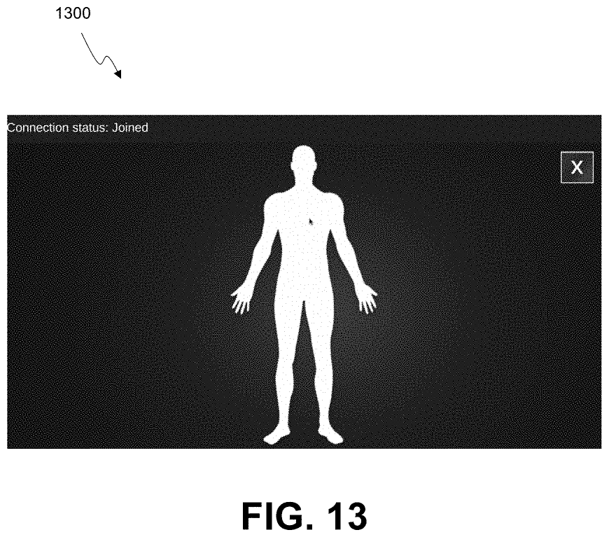

FIG. 13 illustrates an emotion location screen 1300, according to one or more embodiments. Emotion location screen 1300 may present to a session master or a session guest an ability to select a physical location at which a given emotion is felt by the client. For example, emotion location screen 1300 may present an outline of a generic human body upon which a session master or a session guest may click, point, or touch to indicate where on the clients body a given emotion is felt.

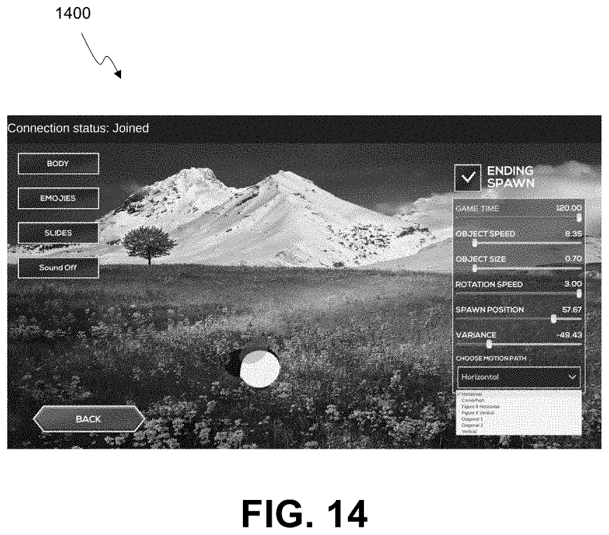

FIG. 14 illustrates a configuration screen 1400, according to one or more embodiments. Configuration screen 1400 may present to a session master or a session guest an ability to control various options and configurations of the motion of a foreground object on a background. For example, the session master or the session guest may select a motion path for the foreground object. Such a motion path may be selected from, inter alia, a horizontal motion path, a corner path, a figure-eight horizontal path, a figure-eight vertical path, a diagonal path, or a vertical path. The motion path may be configured before the session, during the session, and may vary between sets within a session.

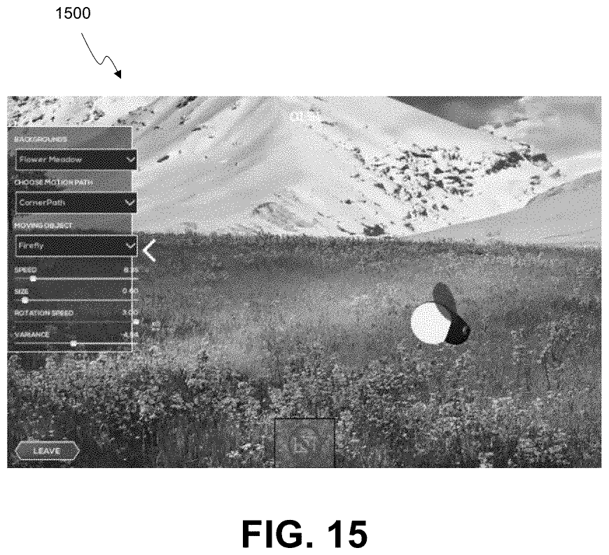

FIG. 15 illustrates an in-session screen 1500, according to one or more embodiments. In-session screen 1500 may include an in-session representation a foreground object traversing a background and options for the in-session configuration of the same. In-session screen 1500 may be configured such that no video feed is shown.

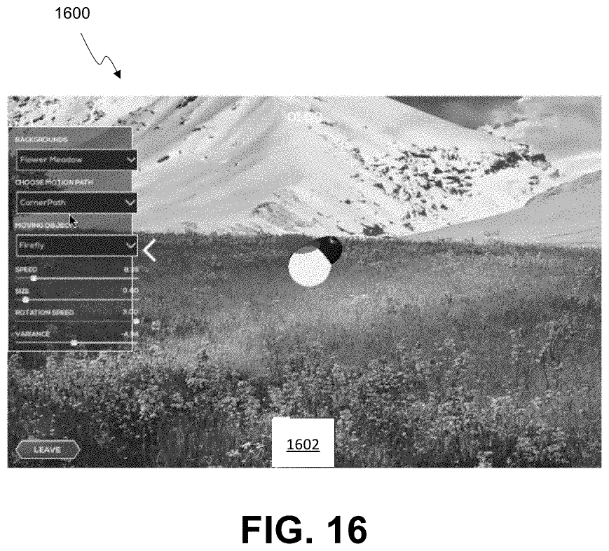

FIG. 16 illustrates an in-session screen 1600, according to one or more embodiments. In-session screen 1600 may represent a foreground object traversing a background and options for the in-session configuration of the same. In-session screen 1600 may be configured such that a video feed 1702, for example, of the client is displayed such that the session master (e.g., the therapist) may observe the client as the client observes the foreground object, its motion, and describes a situation or answers questions in accordance with the particular aspects of the session being directed by the therapist.

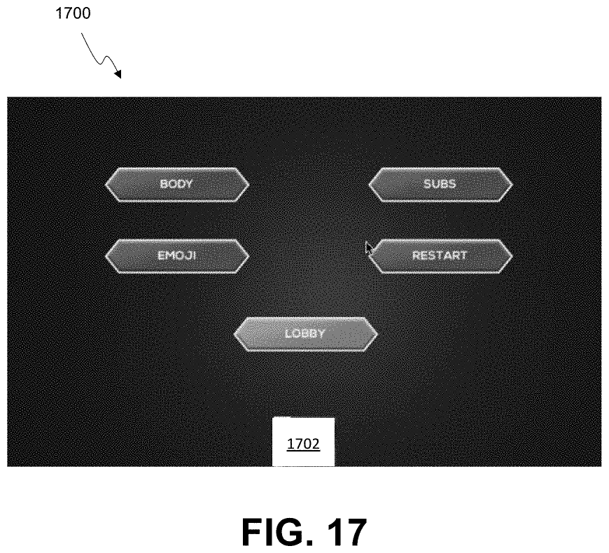

FIG. 17 illustrates a lobby screen 1700, according to one or more embodiments. Lobby screen 1700 may be used by a session master to conduct a post-session screening. Lobby screen 1700 may present options for the session master to input screening observations regarding the client's body, the locations at which the client feels a given emotion, emotions the client is feeling and their levels, a SUDs rating, an ability to restart the session, and ability to return to the lobby. Lobby screen 1700 may be configured such that a video feed 1702, for example, of the client is displayed such that the session master may observe the client as the client observes the foreground object, its motion, and describes or answers in accordance with the particular aspects of the session being addressed.

Various characteristics, advantages, embodiments, and/or examples relating to the invention have been described in the foregoing description with reference to the accompanying drawings. However, the above description and drawings are illustrative only. The invention is not limited to the illustrated embodiments and/or examples, and all embodiments and/or examples of the invention need not necessarily achieve every advantage or purpose, or possess every characteristic, identified herein. Accordingly, various changes, modifications, or omissions may be effected by one skilled in the art without departing from the scope or spirit of the invention. Elements and uses of the above-described embodiments and/or examples can be rearranged and combined in manners other than specifically described above, with any and all permutations within the scope of the invention.

Unless the phrase ‘means for’ or ‘step for’ appears in a particular sample claim or sample claim limitation, such sample claim or sample claim limitation should not be interpreted to invoke 35 U.S.C. § 112(f).

Use of “and” herein to join elements in a list forms a group of all elements of the list. For example, a list described as comprising A, B, and C defines a list that includes A, includes B, and includes C. Use of “or” herein to join elements in a list forms a group of at least one element of the list. For example, a list described as comprising A, B, or C defines a list that may include A, may include B, may include C, may include any subset of A, B, and C, or may include A, B, and C. Unless otherwise stated, lists herein are inclusive, that is, lists are not limited to the stated elements and may be combined with other elements not specifically stated in a list.

The inventors hereby state their intent to rely on the Doctrine of Equivalents to determine and assess the reasonably fair scope of their invention as pertains to any apparatus not materially departing from but outside the literal scope of the invention as set out in the following claims.

We claim:

1. A method, comprising:
connecting a session master to a session;
creating a room;
connecting a session guest to the session;
configuring the room;
conducting a baseline screening;
conducting a session set; and
conducting a post-session screening.

2. The method of claim 1, wherein configuring the room is performed with both the session master and the session guest.

3. The method of claim 1, wherein conducting the baseline screening includes determining an emotional level for a client.

4. The method of claim 1, wherein the room comprises a foreground object and a background, and wherein configuring the room includes configuring a motion path of the foreground object relative to the background.

5. The method of claim 4, wherein the motion path of the foreground object relative to the background includes a horizontal motion path, a corner path motion path, a figure-eight horizontal motion path, a figure-eight vertical motion path, a diagonal motion path, or a vertical motion path.
